(19) United States
(12) Patent Application Publication
(10) Pub. No.: US 2012/0021001 A1
(43) Pub. Date: Jan. 26, 2012

(54) MARKED BOVINE VIRAL DIARRHEA VIRUS VACCINES

(75) Inventors: Chichi Huang, Berwyn, PA (US); Michael G. Sheppard, Victoria (AU); Xuemei Cao, Scituate, MA (US); Gabriele M. Zybarth, Westport, MA (US)

(73) Assignee: PFIZER INC., New York, NY (US)

(21) Appl. No.: 13/165,877

(22) Filed: Jun. 22, 2011

Related U.S. Application Data
(62) Division of application No. 11/995,779, filed on May 13, 2008, now abandoned.
(60) Provisional application No. 60/748,312, filed on Dec. 7, 2005.

(51) Int. Cl. A61K 39/12; C12Q 1/70; C12N 7/00

(52) U.S. Cl. 424/218.1; 435/235.1; 435/5

(57) ABSTRACT

The present invention is directed to a bovine viral diarrhea virus comprising at least one helicase domain amino acid mutation wherein the mutation in the NS3 domain results in a loss of recognition by a monoclonal antibody raised against wild-type NS3 but wherein viral RNA replication and the generation of infectious virus is retained. The present invention is useful, for example, to produce a marked bovine viral diarrhea virus vaccine or to differentiate between vaccinated and infected or unvaccinated animals.

### CROSS REFERENCE TO RELATED APPLICATIONS

Priority is claimed to U.S. Provisional Application Ser. No. 60/748,312, filed Dec. 7, 2005, the disclosure of which is incorporated herein by reference.

### BACKGROUND OF THE INVENTION

Bovine viral diarrhea virus (BVD virus or BVDV) is a small RNA virus of the genus Pestivirus, and family Flaviviridae. It is closely related to viruses which are the causative agents of border disease in sheep and classical swine fever in pigs. Disease caused by BVDV is widespread, and can be economically devastating. BVDV infection can result in breeding problems in cattle, and can cause abortions or premature births. BVDV is capable of crossing the placenta of pregnant cattle, and may result in the birth of persistently infected (PI) calves which are immunotolerant to the virus and persistently viremic for the rest of their lives. (Malmquist, J. Am. Vet. Med. Assoc. 152:763-768 (1968); Ross, et al., J. Am. Vet. Med. Assoc, 188:618-619 (1986)). Infected cattle can also exhibit “mucosal disease”, characterized by elevated temperature, diarrhea, coughing and ulcerations of the alimentary mucosa (Olafson, et al., Cornell Vet. 36:205-213 (1946); Ramsey, et al., North Am. Vet. 34:629-633 (1953)). These persistently infected animals provide a source for dissemination of virus within the herd for further outbreaks of mucosal disease (Liess, et al., Dtsch. Tieraerztl. Wschr. 81:481-487 (1974)) and are highly predisposed to infection with microorganisms responsible for causing enteric diseases or pneumonia (Barber, et al., Vet. Rec. 117:459-464 (1985)).

BVD viruses are classified into one of two biotypes. Those of the “cp” biotype induce a cytopathic effect on cultured cells, whereas viruses of the “ncp” biotype do not (Gillespie, et al., Cornell Vet. 50:73-79 (1960)). In addition, two major genotypes (type 1 and 2) are recognized, both of which have been shown to cause a variety of clinical syndromes (Pellerin, et al., Virology 203:260-268 (1994); Ridpath, et al., Virology 205:66-74(1994)). BVD virions are 40 to 60 nm in diameter. The nucleocapsid of BVDV consists of a single molecule of RNA and the capsid protein C. The nucleocapsid is surrounded by a lipid membrane with two glycoproteins anchored in it, E1 and E2. A third glycoprotein, E^{ms}, is loosely associated to the envelope. The genome of BVDV is approximately 12.5 kb in length, and contains a single open reading frame located between the 5′ and 3′ non-translated regions (NTRs) (Collett, et al., Virology 165:191-199 (1988)). A polyprotein of approximately 438 kD is translated from this open reading frame, and is processed by cellular and viral proteases into at least eleven viral structural and nonstructural (NS) proteins (Tautz, et al., J. Virol. 71:5415-5422 (1997); Xu, et al., J. Viral. 71:5312-5322 (1997); Elbers, et al., J. Virol. 70:4131-4135 (1996); and Wiskerchen, et al., Virology 184:341-350 (1991)). The genomic order of BVDV is p20/N^{pro}, p14/C, gp48/E^{ms}, gp25/E1, gp53/E2, p54/NS2, p80/NS3, p10/NS4A, p32/NS4B, p58/NS5A and p75/NS5B. P20/N^{pro} (Stark, et al., J. Virol. 67:7088-7093 to (1993); Wiskerchen, et al., Virol. 65:4508-4514 (1991)) is a cis-acting, papain-like protease that cleaves itself from the rest of the synthesized polyprotein. The capsid protein (C), also referred to as p14, is a basic protein, and functions in packaging of the genomic RNA and formation of the enveloped virion. P14/C is conserved across different pestiviruses. The three envelope proteins, gp48/E^{ms}, gp25/E1 and gp53/E2, are heavily glycosylated. E^{ms} forms homodimers, covalently linked by disulfides. The absence of a hydrophobic membrane anchor region suggests that E^{ms} is loosely associated with the envelope. E^{ms} induces high antibody titers in infected cattle, but the antisera has limited virus-neutralizing activity. E1 is found in virions covalently linked to gp53/E2 via disulfide bonds. E1 contains two hydrophobic regions that serve to anchor the protein in the membrane, and as a signal peptide for initiating translocation, E1 does not induce a significant antibody response in infected cattle, suggesting that it may not be exposed on the virion's surface. Like E1, E2 also has a membrane anchor region at its C-terminus. Unlike E1, however, E2 is very antigenic, being one of the most immunodominant proteins of BVDV. Antibodies binding to E2 can efficiently neutralize a viral infection, suggesting that it may be involved in virus entry. The region of the polyprotein downstream of the structural proteins encodes the nonstructural proteins, and is processed by two viral proteolytic enzymes. The NS2-NS3 junction is cleaved by a zinc-dependent protease encoded within NS2. The C-terminal portion of the BVDV polyprotein encoding NS3, NS4A, NS4B, NS5A and NS5B is processed by a serine protease encoded by the N-terminal domain of NS3. NS3 is another major BVDV immunogen, as infected cattle develop a strong humoral response to it. In contrast, no serum antibodies are found to the other nonstructural proteins in BVDV-infected cattle, and only a weak humoral immune response to NS4A can be detected. NS3 is found exclusively in cytopathic BVDV isolates, and the region encoding the protein is one of most conserved in the BVDV genome, based on comparisons among BVDV subtypes and other pestiviruses. The C-terminal portion of NS3 encodes a RNA-dependent NTPase/helicase, and based on sequences comparisons of highly conserved helicase amino acid motifs, the BVDV helicase has been classified into the helicase superfamily-2 (SF2). Within this superfamily are similar proteins from the poty-, flavi-, and pestiviruses, including hog cholera (classical swine fever) virus NS3 helicase, and RNA helicases from other flaviviruses, such as West Nile virus, yellow fever virus, hepatitis C virus (HCV) and Japanese encephalitis virus. The molecular structure of the protease and helicase domains of HCV NS3 have been solved (Yao, et al Nat Struct Biol. 4:463-7 (1997); Jin and Peterson, Arch Bioxchem Biophys 323:47-53 (1995)). The protease domain contains the dual β-barrel fold that is commonly seen among members of the chymotrypsin serine protease family. The helicase domain contains two structurally related β-α-β subdomains, and a third subdomain of seven helices and three short β strands, usually referred to as the helicase α-helical subdomain. The nucleoside triphosphate (NTP) and RNA-binding sites, as well as the helicase active site, are surface-exposed, whereas the protease active site is not, and is oriented facing the helicase domain. The protease and helicase domains are covalently connected by a short surface-exposed strand, and interact over a large surface area (˜900 Å²). The helicase active site, however, is oriented away from this area of interaction.

Among the BVDV vaccines currently available are those which contain chemically-inactivated wild-type virus (McClurkin, et al., Arch. Virol. 58:119 (1978); Fernelius, et al., Am. J. Vet. Res. 33:1421-1431 (1972); and Kolar, et al., Am. J. Vet. Res. 33:1415-1420 (1972)). These vaccines typically require the administration of multiple doses, and result in a short-lived immune response; they also do not protect against fetal transmission of the virus (Bolin, Vet. Clin. North Am. Food Anim. Pract. 11:615-625 (1995)). In sheep, a subunit vaccine based on a purified E2 protein has been reported (Bruschke, et al., Vaccine 15:1940-1945 (1997)). Although this vaccine appears to protect fetuses from becoming infected, protection is limited to only the homologous strain of virus, and there is no correlation between antibody titers and protection. Modified live virus (MLV) BVDV vaccines have been produced using virus that has been attenuated by repeated passaging in bovine or porcine cells (Coggins, et al., Cornell Vet. 51:539 (1961); and Phillips, et al., Am. J. Vet. Res. 36:135 (1975)), or by chemically-induced mutations that confer a temperature-sensitive phenotype on the virus (Lobmann, et al., Am. J. Vet. Res. 45:2498 (1984); and Lobmann, et al., Am. J. Vet. Res. 47:557-561 (1986)). A single dose of a MLV BVDV vaccine has proven sufficient for providing protection from infection, and the duration of immunity can extend for years in vaccinated cattle (Coria, et al., Can. J. Con. Med. 42:239 (1978)). In addition, cross-protection has been reported using MLV vaccines (Martin, et al., In “Proceedings of the Conference of Research Workers in Animal Diseases”, 75:183 (1994)). Safety considerations, however—including fetal transmission of the vaccine strain—are a major concern with respect to use of these modified live viral vaccines (Bolin, Vet. Clin. North Am. Food Anim. Pract. 11:615-625 (1995)).

Based on the above, it is clear that a need exists for new and more effective vaccines to control the spread of BVDV, Such a vaccine could be invaluable in future national or regional BVDV eradication programs, and could also be combined with other marked cattle vaccines, representing a substantial advance in livestock farming. One such vaccine is a “marked” vaccine. Such a vaccine lacks an antigenic determinant present in wild-type virus, Animals infected with the wild-type virus mount an immune response to the “marker” immunogenic determinant, while non-infected, vaccinated animals do not, as a result of the determinant not being present in the marked vaccine. Through the use of an immunological assay directed against the marker determinant, infected animals could be differentiated from vaccinated, non-infected animals. By culling out the infected animals, the herd could, over time, become BVD-free. In addition to the benefit of removing the threat of BVD disease, certification of a herd as BVD-free has direct freedom of trade economic benefits.

### SUMMARY OF THE INVENTION

The present invention is directed to a bovine viral diarrhea virus comprising at least one helicase domain amino acid mutation wherein the mutation in the NS3 domain results in a loss of recognition by a monoclonal antibody raised against wild-type NS3 but wherein viral RNA replication and the generation of infectious virus is retained.

The present invention is also directed to a novel marked bovine viral diarrhea virus vaccine comprising a bovine viral diarrhea virus having at least one helicase domain amino acid mutation, wherein NS3 is not recognized by a standard monoclonal antibody to NS3, such as, for example, 20.10.6; 1.11.3; 21.5.8; and 24.8, but wherein viral RNA replication and generation of infectious virus is retained.

The present invention is also directed to an assay for determining whether an animal has been vaccinated, or is unvaccinated or infected with BVDV.

In one embodiment of the present invention, a bovine viral diarrhea virus comprising at least one helicase domain amino acid mutation wherein the mutation in the helicase domain of NS3 results in a loss of recognition by a monoclonal antibody raised against NS3 from wild-type bovine viral diarrhea virus but wherein viral RNA replication and the generation of infectious virus is retained is provided.

In another embodiment of the present invention, a bovine viral diarrhea virus comprising at least one helicase domain amino acid mutation wherein NS3 is not recognized by a monoclonal antibody to NS3, and wherein the NS3 antibody is selected from the group consisting of 20.10.6; 1.11.3; 21.5.8; and 24.8 but wherein viral RNA replication and the generation of infectious virus is retained is provided.

In another embodiment of the invention, the virus vaccine comprises a single helicase domain amino acid mutation.

In another embodiment of the present invention, the virus vaccine comprises a helicase domain mutation within the IGR loop.

In another embodiment of the present invention, the bovine viral diarrhea virus comprises a helicase domain mutation within the IGR loop at amino acid residue 1841.

In another embodiment of the present invention, the bovine viral diarrhea virus comprises a helicase domain mutation within the IGR loop at amino acid residue 1843.

In another embodiment of the present invention, the bovine viral diarrhea virus comprises a helicase domain mutation within the IGR loop at amino acid residue 1845.

In another embodiment of the present invention, the bovine viral diarrhea virus comprises a helicase domain mutation within the KHP loop.

In another embodiment of the present invention, the bovine viral diarrhea virus comprises a helicase domain mutation within the KHP loop at amino acid residue 1867.

In another embodiment of the present invention, the bovine viral diarrhea virus comprises a helicase domain mutation within the KHP loop at amino acid residue 1868.

In another embodiment of the present invention, the bovine viral diarrhea virus comprises a helicase domain mutation within the KHP loop at amino acid residue 1869.

In another embodiment of the present invention, the bovine viral diarrhea virus comprises a helicase domain mutation within the SES loop.

In another embodiment of the present invention, the bovine viral diarrhea virus comprises a helicase domain mutation within the SES loop at amino acid residue 1939.

In another embodiment of the present invention, the bovine viral diarrhea virus comprises a helicase domain mutation within the SES loop at amino acid residue 1942.

In another embodiment of the present invention, the bovine viral diarrhea virus comprises two, three, or four helicase domain amino acid mutations.

In another embodiment of the present invention, the bovine viral diarrhea virus comprises two helicase domain mutations.

In another embodiment of the present invention, the bovine viral diarrhea virus comprises two helicase domain mutations within the IGR loop.

In another embodiment of the present invention, the bovine viral diarrhea virus comprises two helicase domain mutations within the IGR loop at amino acid residues 1843 and 1845.

In another embodiment of the present invention, the bovine viral diarrhea virus comprises two helicase domain mutations within the SES loop.

In another embodiment of the present invention, the bovine viral diarrhea virus comprises two helicase domain mutations within the SES loop at amino acid residues 1939 and 1942.

In another embodiment of the present invention, the bovine viral diarrhea virus comprises three helicase domain mutations.

In another embodiment of the present invention, the bovine viral diarrhea virus comprises three helicase domain mutations within the IGR loop.

In another embodiment of the present invention, the bovine viral diarrhea virus comprises three helicase domain mutations within the IGR loop at amino acid residues 1867, 1868, and 1869.

In another embodiment of the present invention, the bovine viral diarrhea virus comprises three helicase domain mutations within the IGR and the SES loop at amino acid residues 1845, 1868, and 1939.

In one particularly preferred embodiment of the present invention, a marked bovine viral diarrhea virus vaccine is provided, comprising a bovine viral diarrhea virus comprising at least one helicase domain amino acid mutation wherein the mutation in the helicase domain of NS3 results in a loss of recognition by a monoclonal antibody raised against NS3 from wild-type bovine viral diarrhea virus but wherein viral RNA replication and the generation of infectious virus is retained.

In another embodiment of the present invention, a method of differentiating an animal infected with bovine diarrhea virus from an animal vaccinated with a bovine diarrhea virus vaccine is provided. In this embodiment, the bovine diarrhea virus vaccine is a marked vaccine comprising at least one helicase domain amino acid mutation, and the method comprises;

obtaining a test sample from a test animal;

detecting bovine diarrhea virus in the test sample; and

determining whether the bovine diarrhea virus contains the mutation.

In another embodiment of the present invention, the method of detecting bovine diarrhea virus employs the use of at least one monoclonal antibody.

A preferred method comprises a marked vaccine helicase domain amino acid mutation in the helicase domain of NS3.

For example, and embodiment of this differential assay may include the steps of:

adding labeled antibody capable of detecting wild type bovine diarrhea virus or capable of detecting mutated bovine diarrhea virus to a test sample, wherein the test sample contains body fluid from test animal and;

measuring the binding affinity of the labeled antibody to the wild type bovine diarrhea virus or to the mutated bovine diarrhea virus by contacting at least one monoclonal antibody to the wild type bovine diarrhea virus or to the mutated bovine diarrhea virus; and

determining the vaccination status of test animal by comparing results of binding affinity using a monoclonal antibody directed to wild type BVDV versus BVDV with mutated NS3.

A preferred method comprises adding a labeled first antibody directed to a domain other than mutated NS3; and

adding a labeled second antibody directed to a mutated portion of NS3.

In one embodiment of this method, the first antibody is directed to a wild type virus.

In another embodiment of this method, the second antibody is directed to the mutated portion of NS3.

In another embodiment of this method, the second antibody is directed against NS3 and is selected from the group consisting of 20.10.6; 1.11.3; 21.5.8; and 24.8.

In another embodiment of the method, the second antibody is directed to at least one mutated portion of the NS3 selected from the group consisting of the IGR loop, the KHP loop, and the SES loop.

In another embodiment of this method, the bovine viral diarrhea virus comprises at least one helicase domain amino acid mutation within the IGR loop at an amino acid residue selected from the group consisting of 1841, 1843, and 1845.

In another embodiment of the method, the bovine viral diarrhea virus comprises at least one helicase domain amino acid mutation within the KHP loop at an amino acid residue selected from the group consisting of 1867, 1868, and 1869.

In another embodiment if the method, the bovine viral diarrhea virus comprises at least one helicase domain amino acid mutation within the SES loop at an amino acid residue selected from the group consisting of 1939, and 1942.

In another embodiment of the method, the bovine viral diarrhea virus comprises at least one helicase domain amino acid mutation within the IGR loop and the SES loop at amino acid residues 1845, 1868, and 1939.

### BRIEF DESCRIPTION OF THE DRAWINGS

These and other features, aspects and advantages of the present invention are illustrated with reference to the following description, appended claims, and accompanying drawings where

FIG. 1 depicts the domains of NS3.

FIG. 2 shows the sequence alignment of BVDV and HCV helicase domains. The sequence labeled BVDV-NADL is SEQ. ID. NO.: 20. The sequence labeled HCV-BK is SEQ ID. NO.: 21.

FIG. 3 shows an illustration of the molecular model of BVDV helicase

FIG. 4 shows the location of scanning mutants. The sequence labeled BVDV-NADL is SEQ. ID. NO.: 20. The sequence labeled HCV-BK is SEQ ID, NO.: 21.

FIG. 5 shows the domain map of the complete full length BVDV precursor and the BVDV subviral replicon structure.

### BRIEF DESCRIPTION OF THE SEQUENCES

SEQ ID NO. 1 is a peptide sequence of a full length, unprocessed polyprotein from bovine viral diahrrea virus. The numbering of the residues in this sequence corresponds to the mutations described herein. For example, a mutation described as “K1845A” means that the Lysine residue at position 1845 of SEQ ID NO. 1 has been replaced by an Alanine residue;

SEQ ID NO. 2 is a sequence of a DNA plasmid fragment that flanks the 5′ end of p15aDI cloning site for generating exemplary mutants;

SEQ ID NO. 3 is a sequence of a DNA plasmid fragment that flanks the 3′ end of p15aDI cloning site for generating exemplary mutants;

SEQ ID NO. 4 is a sequence of a DNA 5′ primer for introducing the 11841A mutation described herein;

SEQ ID NO. 5 is a sequence of a DNA 3′ primer for introducing the 11841A mutation described herein;

SEQ ID NO. 6 is a sequence of a DNA 5′ primer for introducing the R1843A mutation described herein;

SEQ ID NO. 7 is a sequence of a DNA 3′ primer for introducing the R1843A mutation described herein;

SEQ ID NO. 8 is a sequence of a DNA 5′ primer for introducing the K1845A mutation described herein;

SEQ ID NO. 9 is a sequence of a DNA 3′ primer for introducing the K1845A mutation described herein;

SEQ ID NO. 10 is a sequence of a DNA 5 primer for introducing the K1867A mutation described herein;

SEQ ID NO. 11 is a sequence of a DNA 3′ primer for introducing the K1867A mutation described herein;

SEQ ID NO. 12 is a sequence of a DNA 5′ primer for introducing the H1868A mutation described herein;

SEQ ID NO. 13 is a sequence of a DNA 3′ primer for introducing the H1868A mutation described herein;

SEQ ID NO. 14 is a sequence of a DNA 5′ primer for introducing the P1869A mutation described herein;

SEQ ID NO. 15 is a sequence of a DNA 3′ primer for introducing the P1869A mutation described herein;

SEQ ID NO. 16 is a sequence of a DNA 5′ primer for introducing the E1939A mutation described herein;

SEQ ID NO. 17 is a sequence of a DNA 3′ primer for introducing the E1939A mutation described herein;

SEQ ID NO. 18 is a sequence of a DNA 5′ primer for introducing the R1942A mutation described herein;

SEQ ID NO. 19 is a sequence of a DNA 3′ primer for introducing the R1942A mutation described herein;

SEQ ID NO. 20 is a peptide sequence of domains 1 (helicase) and 2 (NTPase) of the NS3 region of translated BVD; and

SEQ ID NO. 21 is a peptide sequence of domains 1 (helicase) and 2 (NTPase) of the NS3 region of translated Hepatitis C virus (HM).

### Definitions

The following definitions may be applied to terms employed in the description of embodiments of the invention. The following definitions supercede any contradictory definitions contained in each individual reference incorporated herein by reference. Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

The term “amino acid,” as used herein, refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, carboxyglutamate, and O-phosphoserine. Stereoisomers (e.g., D-amino acids) of the twenty conventional amino acids, unnatural amino acids such as α and α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids. Amino acid analogs refer to compounds that have the same basic chemical structure as a naturally occurring amino acid, ie., a carbon that is bound to a hydrogen, a carboxyl group, an amino group, and an R group. Exemplary amino acid analogs include, for example, homoserine, norleucine, methionine sulfoxide, and methionine methyl sulfonium. Such analogs have modified R groups (e.g., norleucine) or modified peptide backbones, but retain the same essential chemical structure as a naturally occurring amino acid. Amino acid mimetics refer to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission.

| Amino Acids-Single | Three letter | |
|---|---|---|
| letter codes: | codes: | Full names |
| G: | Gly: | glycine |
| V: | Val: | valine |
| L: | Leu: | leucine |
| A: | Ala: | alanine |
| I: | Ile: | isoleucine |
| S: | Ser: | serine |
| D: | Asp: | aspartic acid |
| K: | Lys: | lysine |
| R: | Arg: | arginine |
| H: | His: | histidine |
| F: | Phe: | phenylalanine |
| Y: | Tyr: | tyrosine |
| T: | Thr: | threonine |
| C: | Cys: | cysteine |
| M: | Met: | methionine |
| E: | Glu: | glutamic acid |
| W: | Trp: | tryptophan |
| P: | Pro: | proline |
| N: | Asn: | asparagine |
| Q: | Gln: | glutamine |
| X: | Xaa | unspecified amino acid |

The term “animal subjects,” as used herein, is meant to include any animal that is susceptible to BVDV infections, such as bovine, sheep and swine. By “treating” or “vaccinating” is meant preventing or reducing the risk of infection by a virulent strain of BVDV, ameliorating the symptoms of a BVDV infection, or accelerating the recovery from a BVDV infection.

BVD “viruses”, “viral isolates” or “viral strains” as used herein refer to BVD viruses that consist of the viral genome, associated proteins, and other chemical constituents (such as lipids). Ordinarily, the BVD virus has a genome in the farm of RNA. RNA can be reverse-transcribed into DNA for use in cloning. Thus, references made herein to nucleic acid and BVD viral sequences encompass both viral RNA sequences and DNA sequences derived from the viral RNA sequences. For convenience, genomic sequences of BVD as depicted in the SEQUENCE LISTING hereinbelow refer to the polypeptide sequence, and primer DNA sequences used in making the exemplary mutations. The corresponding RNA sequence for each is readily apparent to those of skill in the art.

A number of type I and type II BVD viruses are known to those skilled in the art and are available through, e.g., the American Type Culture Collection.

The term “conservative amino acid substitutions,” as used herein, are those that generally take place within a family of amino acids that are related in their side chains. In particular, as used herein, a conservative amino acid substitution is one that has no effect on antibody recognition of a given peptide as compared with the wild-type derived peptide. Genetically encoded amino acids are generally divided into four groups: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan and tyrosine are also jointly classified as aromatic amino acids.

Accordingly, the term “non-conservative amino acid substitutions,” as used herein, are those that are likely to have different properties, particularly with respect to antibody recognition. Thus, a non-conservative amino acid substitution will evoke a differential immune response, such as, for example, loss of recognition by an antibody raised against a wild-type derived peptide.

The term “immunogenic,” as used herein, means the capacity of a mutated or wild-type BVD virus in provoking an immune response in an animal against type I or type II BVD viruses, or against both type I and type II BVD viruses. The immune response can be a cellular immune response mediated primarily by cytotoxic T-cells, or a humoral immune response mediated primarily by helper T-cells, which in turn activates B-cells leading to antibody production.

As used herein, the term “naked DNA” refers to a plasmid comprising a nucleotide sequences encoding an agent of the present invention together with a short promoter region to control its production. It is called “naked” DNA because the plasmids are not carried in any delivery vehicle. When such a DNA plasmid enters a host cell, such as a eukaryotic cell, the proteins it encodes are transcribed and translated within the cell.

The term “plasmid” as used herein refers to any nucleic acid encoding an expressible gene and includes linear or circular nucleic acids and double or single stranded nucleic acids. The nucleic acid can be DNA or RNA and may comprise modified nucleotides or ribonucleotides, and may be chemically modified by such means as methylation or the inclusion of protecting groups or cap—or tail structures.

The term “vaccine” as used herein refers to a composition which prevents or reduces the risk of infection or which ameliorates the symptoms of infection. The protective effects of a vaccine composition against a pathogen are normally achieved by inducing in the subject an immune response, either a cell-mediated or a humoral immune response or a combination of both. Generally speaking, abolished or reduced incidences of BVDV infection, amelioration of the symptoms, or accelerated elimination of the viruses from the infected subjects are indicative of the protective effects of a vaccine composition. The vaccine compositions of the present invention provide protective effects against infections caused by BVD viruses.

The term “vector,” as used herein, means a tool that allows or facilitates the transfer of a nucleic acid from one environment to another. In accordance with the present invention, and by way of example, some vectors used in recombinant DNA techniques allow nucleic acids, such as a segment of DNA (such as a heterologous DNA segment, for example, a heterologous cDNA segment), to be transferred into a host or a target cell for the purpose of replicating the nucleic acids and/or expressing proteins encoded by the nucleic acids. Examples of vectors used in recombinant DNA techniques include but are not limited to plasmids, chromosomes, artificial chromosomes and viruses.

### DETAILED DESCRIPTION OF THE INVENTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference.

Standard procedures can be used to propagate and purify a plasmid useful in the present invention. The preferred prokaryotic host cell for plasmid propagation is E. coil GM2163 cell line, but some other cell types can also be used. RNA transcribed from the plasmid can be introduced by transfection into eukaryotic host cells capable of supporting virus production, such as MDBK cells. The virus can be produced in such host cells and isolated therefrom in highly purified form using known separation techniques such as sucrose gradient centrifugation.

In one embodiment, the present invention provides immunogenic compositions in which one or more of the mutant BVD viruses described above have been included.

Another embodiment of the present invention is directed to isolated genomic nucleic molecules of the mutant BVD viruses as described above. Nucleic acid molecules as used herein encompass both RNA and DNA.

In this embodiment, the isolated genomic nucleic molecule of a BVD virus contains a genomic sequence of a type I virus wherein at least a portion of the NS3 domain is mutated in the helicase domain.

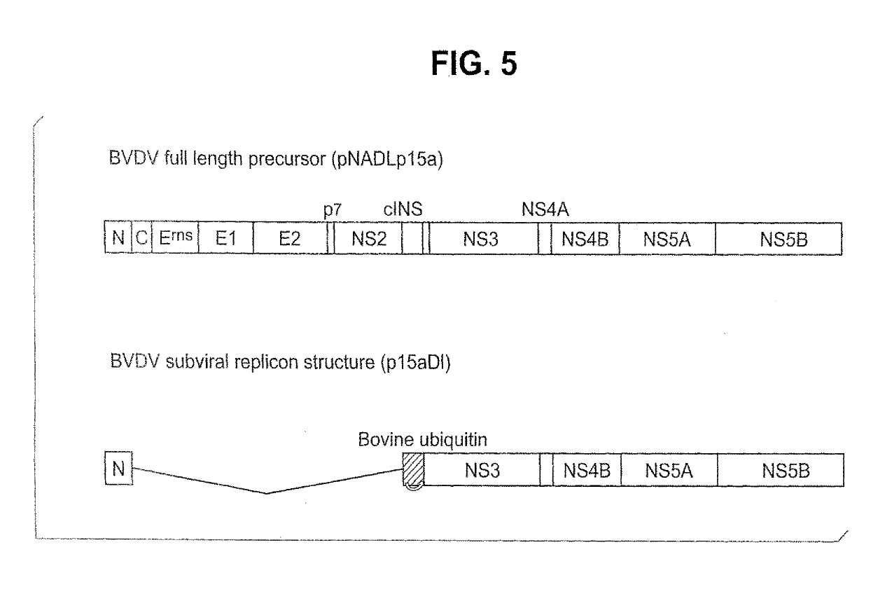

In another embodiment, the present invention provides vectors in which the genomic nucleic acid sequence of a BVD virus as described herein above has been incorporated. Such vectors can be introduced into appropriate host cells, either for the production of large amounts of the genomic nucleic acid molecules or for the production of progeny mutant BVD viruses. The vectors may contain other sequence elements to facilitate vector propagation, isolation and subcloning; for example, selectable marker genes and origins of replication that allow for propagation and selection in bacteria and host cells. A particularly preferred vector of the present invention is p15aDI (see FIG. 5).

Still another embodiment of the present invention is directed to host cells into which the genomic nucleic acid molecule of a mutated BVD virus of the present invention has been introduced. “Host cells” as used herein include any prokaryotic cells transformed with the genomic nucleic acid molecule, preferably provided by an appropriate vector, of a mutated BVD virus. “Host cells” as used herein also include any eukaryotic cells infected with a mutated BVD virus or otherwise carrying the genomic nucleic acid molecule of a mutated BDV virus. A preferred prokaryotic host cell for plasmid propagation is E. coli GM2163 cell line, but other cell types can also be used. Preferred eukaryotic host cells include mammalian cells such as MDBK cells (ATCC CCL 22). However, other cultured cells can be used as well. The invention further includes progeny virus produced in such host cells.

In another embodiment of the present invention, the viruses may be attenuated by chemical inactivation or by serial passages in cell culture prior to use in an immunogenic composition. The methods of attenuation are well known to those skilled in the art.

The immunogenic compositions of the present invention can also include additional active ingredient such as other immunogenic compositions against BVDV, for example, those described in copending U.S. patent application Ser. No. 08/107,908, U.S. Pat. No. 6, 060,457, U.S. Pat. No. 6,015,795, U.S. Pat. No. 6,001,613, and U.S. Pat. No. 5,593,873, all of which are incorporated by reference in their entirety.

In addition, the immunogenic compositions of the present invention can include one or more veterinarily-acceptable carriers. As used herein, “a veterinarily-acceptable carrier” includes any and all solvents, dispersion media, coatings, adjuvants, stabilizing agents, diluents, preservatives, antibacterial and antifungal agents, isotonic agents, adsorption delaying agents, and the like, Diluents can include water, saline, dextrose, ethanol, glycerol, and the like. Isotonic agents can include sodium chloride, dextrose, mannitol, sorbitol, and lactose, among others. Stabilizers include albumin, among others. Adjuvants include, but are not limited to, the RIBI adjuvant system (Ribi inc.), alum, aluminum hydroxide gel, oil-in water emulsions, water-in-oil emulsions such as, e.g., Freund's complete and incomplete adjuvants, Block co polymer (CytRx, Atlanta Ga.), SAF-M (Chiron, Emeryville Calif.), AMPHIGEN® adjuvant, saponin, Quil A, QS-21 (Cambridge Biotech Inc., Cambridge Mass.), or other saponin fractions, monophosphoryl lipid A, Avridine lipid-amine adjuvant, heat-labile enterotoxin from E. coli (recombinant or otherwise), cholera toxin, or muramyl dipeptide, among many others. The immunogenic compositions can further include one or more other immunomodulatory agents such as, e.g., interleukins, interferons, or other cytokines.

The immunogenic compositions of the present invention can be made in various forms depending upon the route of administration. For example, the immunogenic compositions can be made in the form of sterile aqueous solutions or dispersions suitable for injectable use, or made in lyophilized forms using freeze-drying techniques. Lyophilized immunogenic compositions are typically maintained at about 4° C., and can be reconstituted in a stabilizing solution, e.g., saline or and HEPES, with or without adjuvant.

The immunogenic compositions of the present invention can be administered to animal subjects to induce an immune response against BVD viruses. Accordingly, another embodiment of the present invention provides methods of stimulating an immune response against BVD viruses, by administering to an animal subject an effective amount of an immunogenic composition of the present invention described above.

In accordance with the methods of the present invention, a preferred immunogenic composition for administration to an animal subject includes a mutated BVD virus. An immunogenic composition containing a mutated virus, preferably attenuated by chemical inactivation or serial passage in culture, is administered to a cattle preferably via parenteral routes, although other routes of administration can be used as well, such as e.g., by oral, intranasal, intramuscular, intra-lymph node, intradermal, intraperitoneal, subcutaneous, rectal or vaginal administration, or by a combination of routes.

Immunization protocols can be optimized using procedures well known in the art. A single dose can be administered to animals, or, alternatively, two or more inoculations can take place with intervals of two to ten weeks. The extent and nature of the immune responses induced in the cattle can be assessed by using a variety of techniques. For example, sera can be collected from the inoculated animals and tested for the presence of antibodies specific for BVD viruses, e.g., in a conventional virus neutralization assay. Detection of responding CTLs in lymphoid tissues can be achieved by assays such as T cell proliferation, as indicative of the induction of a cellular immune response. The relevant techniques are well described in the art, e.g., Coligan et al. Current Protocols in Immunology, John Wiley & Sons Inc. (1994).

Another embodiment of the present invention is directed to vaccine compositions.

In one embodiment, the vaccine compositions of the present invention include an effective amount of one or more of the above-described mutated BVD viruses. Purified mutated viruses can be used directly in a vaccine composition, or mutated viruses can be further attenuated by way of chemical inactivation or serial passages in vitro. Typically, a vaccine contains between about 1×10⁶ and about 1×10⁸ virus particles, with a veterinarily acceptable carrier, in a volume of between 0.5 and 5 ml. The precise amount of a virus in a vaccine composition effective to provide a protective effect can be determined by a skilled veterinary physician. Veterinarily acceptable carriers suitable for use in vaccine compositions can be any of those described hereinabove.

In another embodiment, the vaccine compositions of the present invention include the nucleic acid molecule of a mutated virus. Either DNA or RNA molecules encoding all or a part of the BVD virus genome can be used in vaccines. The DNA or RNA molecule can be present in a “naked” form or it can be administered together with an agent facilitating cellular uptake (e.g., liposomes or cationic lipids). The typical route of administration will be intramuscular injection of between about 0.1 and about 5 ml of vaccine. Total polynucleotide in the vaccine should generally be between about 0.1 μ/ml and about 5.0 mg/ml. Polynucleotides can be present as part of a suspension, solution or emulsion, but aqueous carriers are generally preferred. Vaccines and vaccination procedures that utilize nucleic acids (DNA or mRNA) have been well described in the art, for example, U.S. Pat. No. 5,703,055, U.S. Pat. No. 5,580,859, U.S. Pat. No. 5,589,466, all of which are incorporated herein by reference.

The vaccine compositions of the present invention can also include additional active ingredient such as other vaccine compositions against BVDV, for example, those described in U.S. Pat. No. 6,060,457, U.S. Pat. No. 6,015,795, U.S. Pat. No. 6,001,613, and U.S. Pat. No. 5,593,373.

Vaccination can be accomplished by a single inoculation or through multiple inoculations. If desired, sera can be collected from the inoculated animals and tested for the presence of antibodies to BVD virus.

In another embodiment of the present invention, the above vaccine compositions of the present invention are used in treating BVDV infections. Accordingly, the present invention provides methods of treating infections in animal subjects caused by BDV viruses by administering to an animal a therapeutically effective amount of a mutated BVD virus of the present invention.

Those skilled in the art can readily determine whether a genetically engineered virus needs to be attenuated before administration. A mutated virus of the present invention can be administered directly to an animal subject without additional attenuation. The amount of a virus that is therapeutically effective may vary depending on the particular virus used, the condition of the cattle and/or the degree of infection, and can be determined by a veterinarian.

In practicing the present methods, a vaccine composition of the present invention is administered to a cattle preferably via parenteral routes, although other routes of administration can be used as well, such as e.g., by oral, intranasal, intramuscular, intra-lymph node, intradermal, intraperitoneal, subcutaneous, rectal or vaginal administration, or by a combination of routes. Boosting regiments may be required and the dosage regimen can be adjusted to provide optimal immunization.

A further aspect of the present invention provides methods of determining the attenuated virus of a prior vaccination as the origin of the BVD virus present in an animal subject.

The mutant BVD viruses of the present invention are distinguished from wild type BVD strains in both the genomic composition and the proteins expressed. Such distinction allows discrimination between vaccinated and infected animals, and permits the identification of the BVDV in the event of alleged vaccine-associated outbreaks. For example, a determination can be made as to whether an animal tested positive for BVDV in certain laboratory tests carries a virulent or pathogenic BVD virus, or simply carries a mutant BVD virus of the present invention previously inoculated through vaccination.

A variety of assays can be employed for making the determination. For example, the viruses can be isolated from the animal subject tested positive for BVDV, and nucleic acid-based assays can be used to determine the presence of a mutant BVD viral genome as indicative of a BVD virus used in a prior vaccination. The nucleic acid-based assays include Southern or Northern blot analysis, PCR, and sequencing. Alternatively, protein-based assays can be employed. In protein-based assays, cells or tissues suspected of an infection can be isolated from the animal tested positive for BVDV. Cellular extracts can be made from such cells or tissues and can be subjected to, e.g., Western Blot, using appropriate antibodies against viral proteins that may distinctively identify the presence of the mutant virus previously inoculated, as opposed to the presence of wild-type BVDV.

### Forms and Administration

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula I used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(di-lactic-coglycolic)acid (PGLA) microspheres.

### Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Transdermal Penetration Enhancers: Applications, Limitations, and Potential J. Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 10 ng to 100 μg of the compound of formula 1. The overall daily dose will typically be in the range 1 μg to 100 mg which may be administered in a single dose or, more usually, as divided doses throughout the day.

### Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Ocular/Aural Administration

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release, Modified release formulations include delayed-, sustained-, pulsed-, controlled-, targeted, or programmed release.

### Kit-of-Parts

Inasmuch as it may desirable to administer a combination of active compounds, for example, for the purpose of treating a particular disease or condition, it is within the scope of the present invention that two or more pharmaceutical compositions, at least one of which contains a vaccine in accordance with the invention, may conveniently be combined in the form of a kit suitable for co-administration of the compositions.

Thus the kit of the invention comprises two or more separate pharmaceutical compositions, at least one of which contains a vaccine in accordance with the invention, and means for separately retaining said compositions, such as a container, divided bottle, or divided foil packet. An example of such a kit is a syringe and needle, and the like.

The kit of the invention is particularly suitable for administering different dosage forms, for example, oral and parenteral, for administering the separate compositions at different dosage intervals, or for titrating the separate compositions against one another. To assist a veterinarian, the kit typically comprises directions for administration.

The present invention is further illustrated by, but by no means limited to, the following examples.

### EXAMPLES

### Example 1

### Epitope Mapping of NS3 Domains

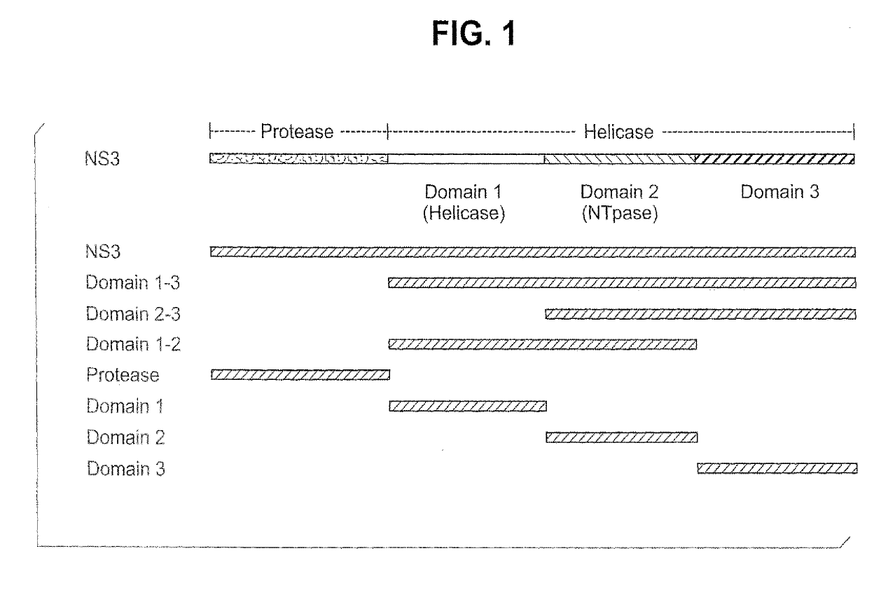

An epitope mapping method was applied to identify the specific epitopes recognized in the NS3 protein by a panel of mAbs. The method entails PCR amplification of each test fragment, followed by translation of the truncated protein in vitro, and finally testing of its reactivity with various mAbs. To preliminarily identify antigenic regions on NS3, a set of seven DNA fragments representing the region were amplified (FIG. 1). Each fragment contained at its 5′ end a T7 promoter followed by an initiation codon, and a stop codon at the 3′ end. These DNA fragments were used as template for the generation of S³⁵-labeled protein fragments by in vitro transcription/translation using the TnT® Rabbit Reticulocyte Lysate System (Promega; Madison, Wis.) and radio-labeled methionine and cysteine. The resulting translated protein fragments included full-length NS3 protein, helicase, and protease, as well as individual subdomains of the helicase. (The boundaries of the protease, helicase and helicase subdomains were identified based on sequence alignment of the BVDV and HCV NS3 proteins,) A panel of 12 mAbs recognizing BVDV NS3, including several used by diagnostic laboratories for the detection of BVDV infection in cattle, were used to immunoprecipitate the translated proteins. These monoclonal antibodies are known in the art, and described as being “previously prepared” in Deregt et al., Mapping of two antigenic domains on the NS3 protein of the pestivirus bovine viral diarrhea virus, Veterinary Microbiology (2005), 108 (1-2), 13-22. The immunoprecipitates were then analyzed by SDS-PAGE and fluorography.

The results of the immunoprecipitation are summarized in Table 1, All 12 mAbs and the polyclonal serum (POLY) recognized full length NS3, and one or more helicase subdomains, while none recognized the protease fragment. Three mAbs (1.11.3, 21.5.8, and 24.8) immunoprecipitated both the full-length helicase and domain 1-domain 2 (d1-d2) fragment but not the d2-d3 fragment, suggesting that these three antibodies recognize domain 1 of the helicase protein. Both mAbs 21.5.8 and 24.8 bound to the dl fragment, but mAb 1.11.3 did not, suggesting that the 1.11.3 antibody was more sensitive to epitope conformation than either of the 21.5.8 and 24.8 mAbs, MAb 2.32.5 recognized both the full length helicase and to some extent the d1-d2 fragment, but not the d2-d3 fragment, suggesting that it may also recognize domain 1. Weak binding of the d1-d2 fragment may indicate that the epitope recognized by 2.32.5 differs between the d1-d2 fragment and full-length helicase. MAbs 4.11.4 and 16.1.5 bound both the full-length NS3 and helicase, but only weakly to the d1-d2 and d2-d3 fragments, suggesting they may be specific for an epitope within the second domain of the helicase, Four mAbs, 5.2.1, 9.10.4, 12.7.3 and 15.14.6 recognize both full-length NS3 and the helicase. They also weakly bound to the d2-d3 fragment, but not the d1-d2 fragment, suggesting that they recognize epitopes located in domain 3. That none of them bound to the single d3 fragment suggests that proper folding of d3 may not occur in the absence of the other subdomains. MAb19.7.6 bound to NS3 and the full-length helicase, but not to any of the other fragments. Recognition by this antibody may require the presence of the intact helicase protein. MAb 20.10.6 bound to NS3, the full-length helicase, and both the d1-d2 and d2-d3 fragments very well. It also recognized the single d2 fragment, suggesting that the epitope in domain 2 recognized by this antibody is not affected by the absence of domains 1 and 3. That none of the 12 mAbs bound to full-length protease was not surprising, as even the polyserum (POLY) from a BVDV-infected cow did not recognize the protease in our experiments, strongly suggesting that the protease is not very antigenic. This is consistent with both the molecular orientation of the protease, helicase, and NS4A (protease cofactor) proteins in HCV, in that the orientation of the protease between the helicase and NS4A proteins leaves very little of its surface structure accessible to antibody binding. Based on these results domain 1 is an exemplary target for introduction of a mutation(s) resulting in a marked virus.

TABLE 1
Immunoprecipitation of NS3 Subdomains

| | 1.11.3 | 2.32.5 | 4.11.4 | 5.2.1 | 9.10.4 | 12.7.3 | 15.14.6 | 16.1.5 | 19.7.6 | 21.5.8 | 24.8 | 20.10.6 | POLY |
|---|---|---|---|---|---|---|---|---|---|---|---|---|---|
| NS3 | + | ++ | ++ | ++ | ++ | + | ++ | + | + | ++ | ++ | ++ | ++ |
| Domain 1-3 | ++ | ++ | ++ | ++ | ++ | ++ | ++ | + | + | ++ | ++ | ++ | ++ |
| Domain 1-2 | ++ | +/− | +/− | − | − | − | − | +/− | − | ++ | ++ | ++ | ++ |
| Domain 2-3 | − | − | +/− | +/− | +/− | +/− | +/− | +/− | − | − | − | ++ | ++ |
| Protease | − | − | − | − | − | − | − | − | − | − | − | − | − |
| Domain 1 | − | − | − | − | − | − | − | − | − | ++ | ++ | − | +/− |
| Domain 2 | − | − | − | − | − | − | − | − | − | − | − | + | + |
| Domain 3 | − | − | − | − | − | − | − | − | − | − | − | − | +/− |
| Epitope | d1 | d1 | d2-d3 | d3 | d3 | d3 | d3 | d2-d3 | d1 | d1 | d1 | d2 | NS3 |

### Example 2

### Sequence Alignment of BVDV and HCV Helicases

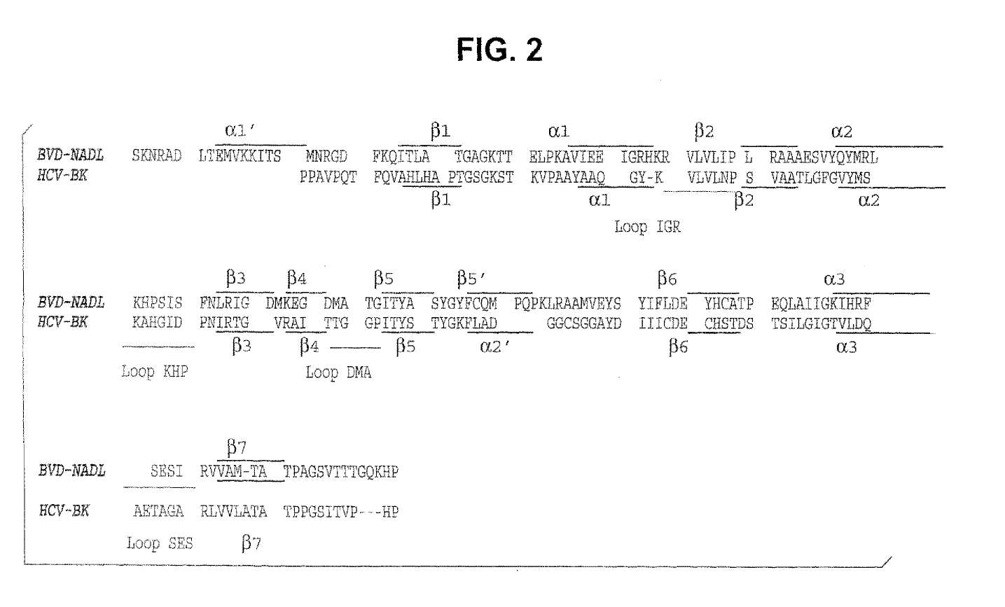

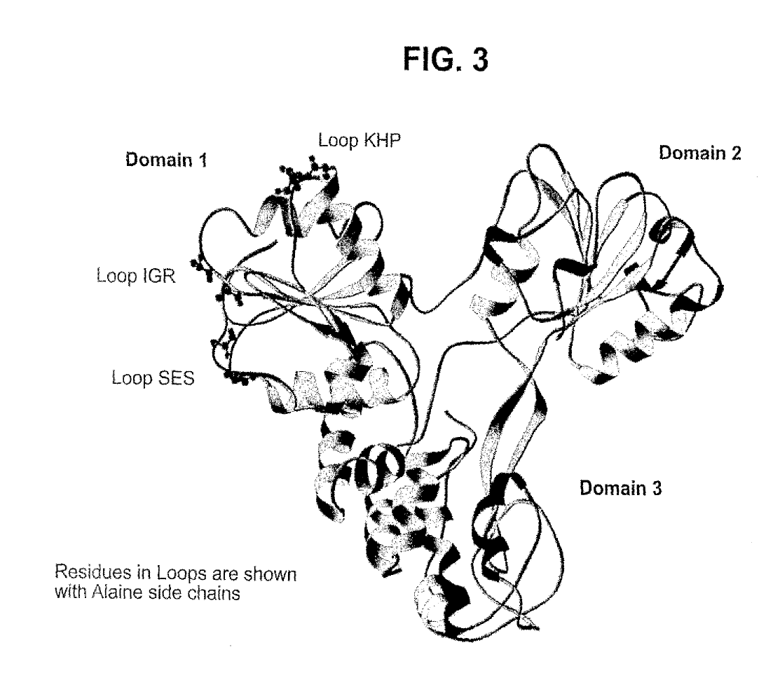

In order to generate a marked virus based on a mutation within domain 1 of the BVDV helicase, further refinement of the epitopes within this domain is desirable. It is desirable to delete an immunodominant epitope without significantly altering the function of the helicase. In order to facilitate the identification of candidate epitopes to mutate, a molecular model of the BVDV helicase would be extremely useful. Since the crystal structure of the HCV helicase is known, it can be used as a template for modeling. To begin the process of generating a molecular model of domain 1, the amino acid sequences of domain 1 of the BVDV and HCV helicases were aligned. The primary sequence identity between them is about 34%. To elucidate the secondary structure of the BVDV helicase domain 1, 47 NS3 sequences derived from various BVDV isolates and other pestivirus were aligned using the Pileup program from the Genetics Computer Group software package (University of Wisconsin; Madison, Wis.), and the NADL BVDV strain as prototypical sequence. From the aligned sequences, a multiple sequence file (MSF) was generated, and submitted to the JPred server (Cuff, et al., Bioinformatics, 14:892-893 (1998)) for secondary structure prediction using the PHD prediction method (Rost and Sander, J. Mol. Biol. 235:13-26 (1993). A Silicon Graphics Indigo2 Impact 10000 workstation (Silicon Graphics; Mountain View, Calif.) was used for all molecular modeling studies. The Molecular Operating Environment (MOE) version 2001.01 (Chemical Computing Group, Inc.; Montreal, Quebec) and SYBYL 6.7 software (Tripes Associates inc.; St. Louis, Mo.) were used for molecular modeling and visualizations. The amino acid sequences of domain 1 and 2 from the HCV (SEQ ID NO. 21) and BVDV (SEQ ID NO. 20) NS3 proteins were aligned (FIG. 2) based on the primary sequence homology and secondary structure predictions. A preliminary molecular model of the BVDV NS3 domain 1 and 2 was then generated, using the corresponding region of the HCV protein as template. As shown in FIG. 3, the presence of several loops and turns between the alpha helices and beta strands, including α1-β2 (Loop IGR), α2-β3 (KHP), β4-β5 (DMA) and α3-β7 (SES), leads to the formation of an exposed surface away from both the helicase catalytic center and the helicase-protease interactive surface. This area has the potential to be a highly antigenic region. Three of the loops identified, Loop KHP, Loop IGP, and Loop SES, were chosen as targets for a mutagenesis study.

### Example 3

### Location of mAb Binding Sites by Scanning Mutagenesis

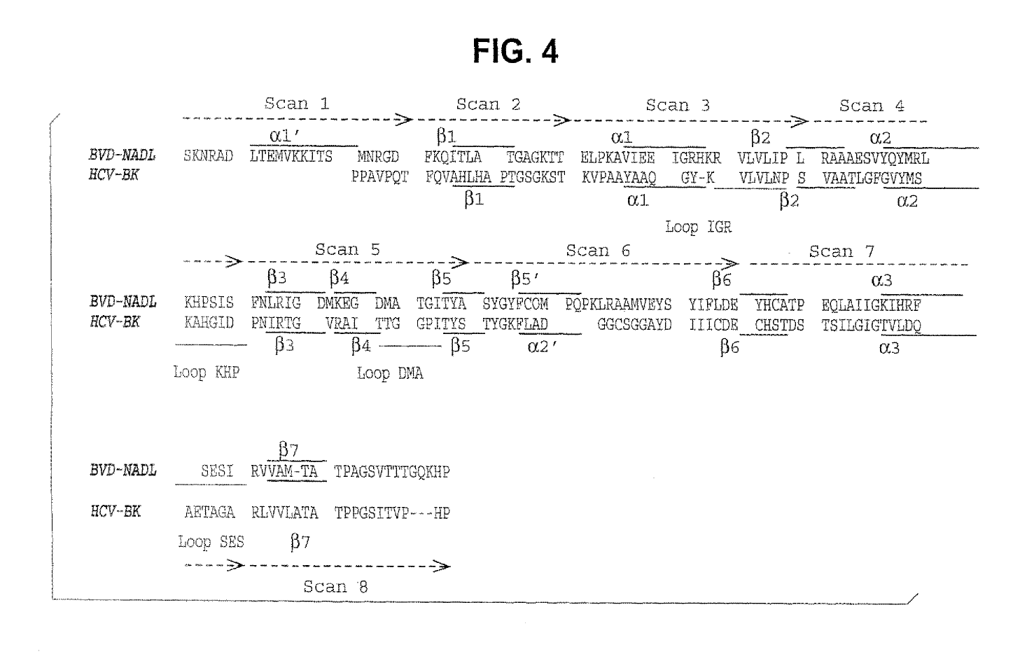

To further define epitopes in domain 1 bound by various mAbs, a scanning mutagenesis method was employed. Briefly, short segments of the BVDV helicase domain 1 sequence (SEQ ID NO. 20) were replaced with the corresponding HCV sequence (SEQ ID NO 21) using PCR amplification, followed by restriction enzyme digestion and ligation of the resulting fragments, generating the “scanning mutants” indicated in FIG. 4. In vitro transcription and translation, as well as immunoprecipitation, was carried out as described in Example 1. A summary of reactivity of the various mAbs with the mutants is shown in Table 2.

TABLE 2
Reactivity of Scanning Mutants with mAbs

| mAbs | Scan | Scan | Scan | Scan | Scan | Scan | Scan | Helicase |
|---|---|---|---|---|---|---|---|---|
| 1.11.3 | ++ | − | ++ | − | − | − | + | +++++ |
| 21.5.8 | ++ | − | +/− | − | +/− | + | ++ | +++++ |
| 24.8 | ++ | + | − | − | − | +/− | ++ | +++++ |
| 20.10.6 | ++++ | +++ | +++++ | ++ | ++ | ++ | ++ | +++++ |
| Poly | +++++ | ++++ | +++++ | ++ | ++ | ++ | +++ | +++++ |
| serum | | | | | | | | |
| CA72 | − | − | − | − | − | − | − | − |
| negativ | | | | | | | | |

### Example 4

### Detailed Resolution of mAb Binding Sites by Alanine Replacement Mutagenesis

To further define the epitopes in domain 1 bound by various mAbs, and to identify the critical residues in these regions for antibody binding, a total of sixteen single amino acid (alanine) replacement mutants in three regions, 11841-R1846, K1867-S1872 and S1938-11941 were generated and tested for antibody binding. Amino acid residue coordinates are according to SEQ ID NO. 1. Thus, “11841A” represents a replacement of Isoleucine with Alanine at coordinate 1841 as numbered in SEQ ID NO. 1. Of course, in other BVDV isolates, different specific amino acids may be present at the particular coordinates of the exemplary sequence. Therefore, a mutation at the same locus of the helicase domain of a variant BVD virus, or plasmid constructed to express a variant BVD virus, will result in an equivalent loss of recognition by antibodies raised against the variant, unmodified virus peptide. The replacement mutants were constructed using a PCR overlap extension technique known in the art (see for example, Ho et al., Gene, 77 (1):51-9 (1989)), Briefly, PCR was used to generate the alanine replacement fragments, each encoding domain 1 and 2 of the helicase. Each fragment encoded a T7 promoter sequence and translation initiation codon at its 5 end, and a stop codon at the 3′ end. Initially, two separate reactions were carried out to generate overlapping fragments encoding the 5′ and 3′ halves of the replacement region. Within the region of overlap, a single alanine mutation was introduced into the sequence of both fragments by virtue of mutagenic oligonucleotide primers used in the PCR. The products of each PCR were separated by electrophoresis in an agarose gel, and a single band of the correct size was purified from each reaction. The purified DNA fragments were mixed and used as templates for a second PCR to generate a single replacement fragment. This entire procedure was repeated to generate each of the desired replacement fragments. The sequence of each fragment was verified by DNA sequencing. S³⁵-labeled protein fragments were generated using these fragments as template via in vitro transcription/translation as described above. Immunoprecipitation using mAbs, followed by SDS-PAGE analysis, was employed to determine if the mutated epitopes were still recognized by the antibodies.

E1939A and R1942A, completely disrupted binding by mAb 1.11.3, suggesting that these two residues are crucial for antibody binding. That these two amino acids are on the same α3-β7 (SES) loop (FIG. 3) suggests that the epitope recognized by this antibody is formed by this loop. Two other mutants, I1841A and K1867A, which are located on two separated regions of the helicase molecule (α1-β2 (IGR) and α2-β3 (KHP) loops), displayed significantly reduced binding by mAb 21.5.8, but not the other antibodies. One conclusion that could be drawn from these results would be that the epitope recognized by this mAb might encompass two different loops which are located in close proximity in the native molecule. This is consistent with the molecular model shown in FIG. 3. The mutant R1843A destroyed binding by mAb 24.8, but had no effect on binding of the other antibodies. Again, this would suggest that this residue is part of a key epitope located on the α1-β2 (IGR) loop. The partial effect of the R1942A mutant on binding of mAb 24.8 suggests that the α3-β7 (SES) loop, together with the α1-β2 (IGR) loop, constitutes the epitope recognized by this antibody. In conclusion, the epitopes recognized by three mAbs were precisely mapped within domain 1 of the BVDV helicase. Key residues within those epitopes were identified, being located within three separate regions of the primary sequence, but in close proximity in the tertiary conformation. The function of these epitopes were further examined in the context of a BVDV subviral replicon.

TABLE 3
Immunoprecipitation of Alanine Replacement Mutants

| | mAb 1.11.3 | mAb 21.5.8 | mAb 24.8 | Poly serum |
|---|---|---|---|---|
| I1841A | + | + | ++ | ++ |
| R1843A | ++ | + | − | ++ |
| H1844A | ++ | + | ++ | ++ |
| K1845A | + | + | ++ | ++ |
| R1846A | ++ | + | ++ | ++ |
| S1938A | ++ | + | ++ | ++ |
| E1939A | − | + | ++ | ++ |
| S1940A | ++ | + | ++ | ++ |
| I1941A | + | + | ++ | ++ |
| R1942A | − | + | +/− | + |
| K1867A | ++ | + | ++ | ++ |
| H1868A | + | + | ++ | ++ |
| P1869A | ++ | + | + | ++ |
| S1870A | ++ | + | ++ | ++ |
| I1871A | ++ | + | ++ | ++ |
| S1872A | ++ | + | ++ | ++ |

### Example 5

### Construction of Helicase Domain 1 Mutations in the Context of a Subviral BVDV Replicon

### Construction of Subviral Replicon

A desireable quality for production of a successful virus vaccine is the ability to obtain high titer virus yields. Therefore, a marker mutation should not interfere significantly with virus replication. As helicase activity is essential for replication of the BVDV RNA, we wanted to assess all domain 1 point mutants made, for not only loss of antibody recognition, but also preservation of catalytic helicase activity. Amplification and genetic manipulation of a full-length BVDV proviral molecular clone in Escherichia coli (E. coli) is difficult because the plasmid is unstable during propagation. Therefore, p15aDI, which contains a truncated subviral replicon expressing NS3 and supporting viral RNA replication, yet lacks the viral structural genes, was created to facilitate screening of the mutants. p15aDI was derived from an infectious proviral parent plasmid (pNADLp15a) containing the full-length BVDV genome. More manipulable because it lacks most of the structural genes and the NS2 coding region, the only sequence located upstream of NS3 consists of a fusion between a portion of the N protein to bovine ubiquitin (FIG. 5). NS3 protein expressed from this replicon is detectable by immunohistochemistry only when efficient RNA replication leads to the amplification of transcripts, resulting in an increase in viral protein expression. Thus, detection of NS3 serves as indirect confirmation of efficient RNA replication and catalytic helicase activity.

### Generation of BVDV Helicase Domain 1 Mutants

A set of twelve different helicase domain 1 mutants were generated in the context of the subviral replicon, and analyzed for viral RNA replication and loss of epitope recognition. Eight of these mutants contained only a single amino acid change, and included: within the IGR loop, I>A (amino acid residue 1841), R>A (1843), and K>A (1845); within the KHP loop, K>A (1867), H>A (1868), and P>A (1869); within the SES loop, E>A (1939), and R>A (1942). Two mutants had changes in two amino acids: within the IGR loop, R>A (1843) and K>A (1845), and within the SES loop, E>A (1939), and R>A (1942). Two contained three changes: K>A (1867), H>A (1868), and P>A (1869), all within the IGR loop, and K>A (1845), H>A (1868), and E>A (1939), affecting multiple loops. While alanine was used in the exemplary mutations for convenience, non-conservative amino acid substitutions may be utilized as appropriate mutations. Each mutant was generated using the overlapping PCR strategy described above. A specific set of overlapping primers was designed for each desired mutation (Table 4). For screening purposes, each primer set also contained additional silent nucleotide changes, which would result in the creation of a unique novel restriction enzyme cleavage site near the site of the mutation. The overlapping PCR fragments served as templates in the second round of amplification, carried out using only the two outside primers. To generate fragments containing multiple amino acid changes, the amplification reaction was repeated, using the previous mutant fragment as template. The fully mutated fragment was then cloned into the subviral replicon backbone by means of two unique restriction enzyme sites (Bsm B I and Sma I) created during the PCR process. The mutant PCR fragment and the subviral replicon backbone were both digested with Bsm B I and Sma I, treated with alkaline phosphatase (NEB, Inc.), purified by agarose gel electrophoresis, and ligated overnight at 16° C. using T4 DNA ligase (New England Biolabs, Inc., Beverly, Mass.). STBL2 E. coli cells (Invitrogen; Carlsbad, Calif.) were transformed with an aliquot of the ligated reaction, and plated on selective media. Colonies were screened by purification of plasmid DNA, followed by digestion with restriction enzymes. Plasmids of the expected size were further confirmed by sequence analysis.

TABLE 4

| SEQ | UTILITY OF | |
|---|---|---|
| ID NO | PRIMER | SEQUENCE (5′-3′) |
| 2 | Flanks 5′ end of | GAGGCCGTTAACATATCA |
| | p15aDI cloning | |
| | site for mutant | |
| | fragments | |
| 3 | Flanks 3′ end of | CCTAAATCACTTTGACCC |
| | p15aDI cloning | TGTTGCTGT |
| | site for mutant | |
| | fragments | |
| 4 | 5′ primer for | GAGGCAGGGCGCCACAAG |
| | introducing | AGAGTATTAGTT |
| | I1841A mutation | |
| 5 | 3′ primer for | CTTGTGGCGCCCTGCCTC |
| | introducing | CTCTATAACTGCTT |
| | I1841A mutation | |
| 6 | 5′ primer for | GAGATAGGCGCCCACAAG |
| | introducing | AGAGTATTAGTT |
| | R184A | |
| | mutation | |
| 7 | 3′ primer for | CTTGTGGGCGCCTATCTC |
| | introducing | CTCTATAAC |
| | R1843A | |
| | mutation | |
| 8 | 5′ primer for | ATAGGGCGCCACGCGAGA |
| | introducing | GTATTAGTTCTTAT |
| | K1845A | |
| | mutation | |
| 9 | 3′ primer for | TCTCGCGTGGCGCCCTAT |
| | introducing | CTCCTTCTATAAC |
| | K1845A | |
| | mutation | |
| 10 | 5′ primer for | TTGGCTCACCCATCGATC |
| | introducing | TCTTTTAACCTAAGGA |
| | K1867A | |
| | mutation | |
| 11 | 3′ primer for | AGAGATCGATGGGTGAGC |
| | introducing | CAATCTCATATACTGGTAG |
| | K1867A | |
| | mutation | |
| 12 | 5′ primer for | AAAGCTCCATCGATCTCT |
| | introducing | TTTAACCTAAGGA |
| | H1868A | |
| | mutation | |
| 13 | 3′ primer for | AGAGATCGATGGAGCTTT |
| | introducing | CAATCTCATATACTGG |
| | H1868A | |
| | mutation | |
| 14 | 5′ primer for | CACGCGAGCATAAGCTTT |
| | introducing | AACCTAAGGATAGGGG |
| | P1869A | |
| | mutation | |
| 15 | 3′ primer for | TTAAAGCTTATGCTCGCG |
| | introducing | TGTTTCAATCTCATATAC |
| | P1869A | |
| | mutation | |
| 16 | 5′ primer for | CCATCGATTTTCAGCGAG |
| | introducing | TATAAGGGTTGTCG |
| | E1939A | |
| | mutation | |
| 17 | 3′ primer for | CTCGCTGAAAATCGATGG |
| | introducing | ATCTTCCCGATAAT |
| | E1939A | |
| | mutation | |
| 18 | 5′ primer for | CCATCGATTTTCAGAGAG |
| | introducing | TATAGCGGTTGTCGCCATGACTGC |
| | R1942A | |
| | mutation | |
| 19 | 3′ primer for | ACCGCTATACTCTCTGAA |
| | introducing | AATCGATGGATCTTCCCGATAAT |
| | R1942A | |
| | mutation | |

### Example 6

### Characterization of Mutant Subviral Replicons

In vitro Transcription and RNA Transfection

RNA transcripts were synthesized in vitro using T7 RNA polymerase and MEGAscript™ (Ambion; Austin, Tex.), DNA templates were linearized with Ksp I and treated with T4 DNA polymerase to remove the 3′ overhang. The products of the transcription reaction were analyzed by agarose gel electrophoresis prior to transfection. 1-5 μg of RNA was added to 200 μl of Opti-MEM (Invitrogen) containing 6 μg of Lipofectin (Invitrogen), and incubated for 10 to 15 min at room temperature. Simultaneously, monolayers (50 to 60% confluent) of Madin Darby Bovine Kidney (MDBK) cells grown in six-well plates (35 mm diameter) were washed twice with RNase-free PBS, and once with Opti-MEM. After the final wash, the transfection mixtures were added to each well, followed by incubation for 10 min at room temperature with gentle rocking. 1 ml of Opti-MEM was then added to each well, and plates were incubated for a further 3 hrs at 37° C. Three ml of Opti-MEM containing 2-3% bovine donor calf serum was then added to each of the wells.

### Analysis of RNA Replication and Antibody Recognition

Following incubation for 24-48 hrs at 37° C., the transfected cells were fixed with 80% acetone, and subjected to an immunohistochemistry assay (IHC), using a Vectastain Elite ABC kit (Vector Laboratories; Burlingame, Calif.) according to the manufacturer's instructions. Monoclonal antibody 20.10.6, which recognizes helicase domain 2, was used to visualize cells positive for NS3, as indicator of efficient RNA replication, Cells transfected with wild-type BVDV RNA, as well as many of the mutant replicons, showed strong staining with mAb 20.10.6, indicating that those individual mutant viral helicases supported efficient vRNA replication. Only mutant K1867A/H1868A/P1869A failed to produce detectable NS3 protein, suggesting that this set of mutations significantly interfered with viral RNA replication.

All cells transfected with wild-type or mutant replicons were also tested with mAbs 1.11.3, 21.5.8, and 24.8. (Table 5). Each loop appeared to be recognized by one of these three antibodies, as mutations in each loop resulted in loss of recognition by one of the three antibodies. In particular, mutation of residues R1843A and K1845A in loop IGR, individually and together, resulted in complete loss of recognition by mAb 24.8. At the same time, recognition by mAbs 20.10.6, 1.11.3 and 21.5.8 was not affected. In loop KHP, mutation K1867A abolished recognition by mAb 21.5.8, without affecting recognition by the other three antibodies. Also, both point mutations in loop SES lead to a loss of recognition by mAb 1.11.3, as did the double mutant, Additionally, the triple mutant (K1845A/H1868A/E1939A) resulted in a loss of recognition by both 1.11.3 and 24,8 mAbs, while antibody recognition by mAbs 20.10.6 and 21.5.8 was not affected.

In summary, several mutations in the three helicase loops that resulted in abolishment of mAb recognition and binding were identified. In addition, it was found that it is feasible to simultaneously disrupt recognition sites for two antibodies, while still maintaining helicase function. Thus, each of these individual mutations, or a combination of them, could serve as a marked BVDV vaccine, containing a mutation(s) within the helicase region.

TABLE 5
Immunoreactivity of mAbs
with Helicase Mutants

| | Monoclonal Antibody | | | |
|---|---|---|---|---|
| Mutation | 20.10.6 | 1.11.3 | 21.5.8 | 24.8 |
| WT/DI | +++ | ++/+++ | ++/+++ | +++ |
| Loop IGR | | | | |
| I1841A | +++ | +++ | +/− | +++ |
| R1843A | +++ | ++ | ++ | − |
| K1845A | +++ | ++/+ | ++ | − |
| RK1843/45A | +++ | ++/+ | +++ | − |
| Loop KHP | | | | |
| K1867A | +++ | ++ | − | + |
| H1868A | +++ | ++ | ++ | ++/+ |
| P1869A | +++ | ++/+++ | +++ | +++ |
| KHP1867/68/69A | − | | | |
| Loop SES | | | | |
| E1939A | +++ | − | ++ | +++ |
| R1942A | +++ | − | ++ | +++ |
| ER1939/42A | +++ | − | +/− | ++/+++ |
| Multiple Loops | | | | |
| K1845A-H1868A-E1939A | +++ | − | | − |
| K1845A-KHP1868FAS- | | | +/++ | |
| ER1939A | | | | |

### Example 7

### Generation and Analysis of Marked Viruses

In order to evaluate the effect(s) of directed mutations within the NS3 protein on viral replication and infectivity, it was necessary to move the mutations into a proviral plasmid containing the full-length BVDV sequence (pNADLp15A). The three mutated sequences chosen for further study were: K1845A-H1868A-E1939A, R1942A, and E1939A. A DNA fragment containing each respective mutated sequence of interest was cloned into pNADLp15A, once again utilizing the unique Bsm BI and Sma I restriction sites. The ligation mixtures were transformed into E. coli GM2163 cells (New England Biolabs, Inc.; Beverly, Mass.), and then plated on selective media. Following overnight incubation, colonies were screened for the presence of plasmid containing the correct sequence. One clone representing each mutation was selected (R1942A; E1939A; and K-H-E), and from these clones, viral RNA was prepared as described in Example 6. MDBK cells were transfected with each RNA preparation, and incubated at 37° C. for 64 hours. Duplicate transfections of RD cells (ATCC; Rockville, Md.) were set up for each mutant. One set of transfected cells was fixed for IHC staining as described in Example 6, and from the second set, cells were scraped from the seeded flasks and stored at −80° C. as stocks for future propagations.

In order to further evaluate the virus produced by the three clones, culture fluids harvested from the transfection experiment were passed onto the fresh RD cell monolayers. Following adsorption and overnight incubation, cells were fixed for HC analysis. The results of that analysis are shown in Table 6. Both the wild-type and mutant viruses were recognized by mAb 20.10.6 (control antibody). The wild-type virus was also recognized by mAbs 1.11.3 and 24.8. Mutant El 939A was bound by mAb 24.8, but not 1.11.3. Mutant K-H-E was recognized only by mAb 20.10.6, and not by 1.11.3 or 24.8. Mutant R1942A demonstrated reactivity with mAb 24.8, but not with 1.11.3.

TABLE 6
IHC Analysis of Cells Infected with Marked Viruses

Monoclonal Antibody

| | | | | |
|---|---|---|---|---|
| Mutation | 20.10.6 | 1.11.3 | 21.5.8 | 24.8 |

| | |
|---|---|
| Loop 2 | |
| K1867A | No Virus Growth |

| | | | | |
|---|---|---|---|---|
| Loop 3 | | | | |
| E1939A | +++ | − | ++ | +++ |
| R1942A | +++ | − | ++ | +++ |
| Multiple Loops | | | | |
| K1845A-H1868A- | +++ | − | +/++ | − |
| E1939A | | | | |

The growth kinetics of each marked virus was also assessed. Stock virus titers for each were pre-determined using a standard virus titration protocol. In a time-course study, fresh monolayers of RD cells were seeded in tissue culture flasks, incubated overnight, and the following day infected with a pre-determined amount of each virus. Following adsorption and washing, an initial set of samples were collected (Hour “0”). Samples were subsequently collected at 14, 19, 24, 39, 43, 47, and 65 hrs post infection, Virus titers were determined using the Spearman-Karber method (Hawkes, R. A. In E. H. Lennette (ed.), Diagnostic Procedures for Viral, Rickettsial and Chlamydial Infections, p. 33-35; 7th ed. American Public Health Association Publications, Washington, D.C.) and expressed as TCID₅₀/ml. Compared to the wild-type (parent) BVD virus, all of the mutants grew at a rate similar to, or in some cases, slightly better than, the wild-type (Table 7).

TABLE 7
Comparative Titers of Wild-Type and
Mutant BVD Viruses (TCID₅₀/ml)

| Hours | Wild Type NDAL | | K-H-E#9 | | R1942A#73 | | E1939A#84 | |
|---|---|---|---|---|---|---|---|---|
| 0 | 0 | 4 | 4 | 2.5 | 0 | 0 | 0 | 0 |
| 14 | 2.5e+3 | 1.6e+3 | 1.0e+1 | 2.5e+1 | 2.5e+2 | 4.0e+2 | 6.3e+2 | 2.5e+3 |
| 19 | 6.3e+3 | 6.3e+3 | 1.0e+3 | 4.0e+3 | 1.6e+3 | 4.0e+3 | 4.0e+3 | 6.3e+3 |
| 24 | 1.6e+4 | 4.0e+4 | N/D | N/D | 1.6e+3 | 6.3e+3 | 2.5e+4 | 2.5e+4 |
| 39 | 4.0e+5 | N/D | N/D | N/D | 6.3e+4 | 1.0e+5 | 1.0e+6 | 4.0e+5 |
| 43 | 2.5e+5 | 6.3e+5 | 6.3e+4 | 6.3e+4 | 1.6e+5 | 1.6e+5 | 1.0e+6 | 2.5e+6 |
| 47 | 1.6e+5 | 5.0e+5 | 1.6e+5 | 2.5e+5 | 2.5e+5 | 4.0e+5 | 1.6e+6 | 4.0e+6 |
| 65 | 1.6e+5 | 2.8e+5 | 4.0e+5 | 2.5e+5 | 2.5e+6 | 2.5e+6 | 6.3e+6 | 1.0e+7 |

Some of the mutations generated resulted in the alteration of specific immunologically distinct epitopes, as determined by a panel of monoclonal antibodies. Similar results were obtained when antibody recognition was analyzed in the context of an infectious viral particle. Clones containing mutations which did not interfere with the generation of infectious virus, yet led to a loss in recognition by mAbs, represent novel strains which serve as effective marked BVDV vaccine strains.

### Example 8

### Vaccine Efficacy Testing in a Young Calf Model

BVDV negative healthy calves are obtained, randomly assigned to study groups, and maintained under supervision of an attending veterinarian. The test vaccine is combined with a sterile adjuvant, and administered by either intramuscular (IM) or subcutaneous (SC) injection. Two doses of vaccine are administered, 21 to 28 days apart. The animals are subsequently challenged at 21 to 28 days following the final vaccination with a Type 1 or Type 2 strain of BVDV. Challenge inoculum is given intranasally in a 4 ml divided dose, 2 ml per nostril. Control groups consisting of unvaccinated, unchallenged animals and/or unvaccinated, challenged animals are also maintained throughout the study.

Clinical parameters are monitored daily, including rectal temperature, depression, anorexia, and diarrhea. Serum neutralization titers are determined by a constant-virus, decreasing-serum assay in bovine cell culture, using serial dilutions of serum combined with a BVDV Type 1 or 2 strain. Post-challenge isolation of BVDV in bovine cell culture is attempted from peripheral blood. A BVDV-positive cell culture is determined by indirect immunofluorescence. To demonstrate protection following challenge, a reduction in incidence of infection has to be demonstrated in vaccinated groups versus the control groups.

### Example 9

### Vaccine Efficacy Testing in a Pregnant Cow-Calf Model

BVDV-negative cows and heifers of breeding age are obtained and randomly assigned to a vaccination test group or a placebo (control) group. Cows are inoculated twice by intramuscular (IM) or subcutaneous (SC) injection, with either vaccine or placebo, 21 to 28 days apart. Following the second vaccination, all cows receive an IM prostaglandin injection to synchronize estrus. Cows which display estrus are bred by artificial insemination with certified BVDV-negative semen. At approximately 60 days of gestation, the pregnancy status of cows is determined by rectal palpation. Approximately 6 weeks later, cows with confirmed pregnancies are randomly selected from each test group. Each of these cows is challenged by intranasal inoculation of BVDV Type 1 or 2. Blood samples are collected on the day of challenge and at multiple postchallenge intervals for purposes of BVDV isolation.

Twenty-eight days after challenge, left flank laparotomies are performed and amniotic fluid is extracted from each cow. Immediately prior to surgery, a blood sample is collected from each cow for serum neutralization assays. Following caesarian delivery, a blood sample is collected from each fetus. Fetuses are then euthanized, and tissues are aseptically collected for purposes of BVDV isolation. In cases where spontaneous abortions occur, blood samples are taken from the dam when abortion is detected and two weeks later. The paired blood samples and aborted fetuses are subjected to serologic testing and virus isolation. Vaccine efficacy is demonstrated by a lack of fetal infection and late-term abortion.

### Example 10

### Diagnostic Assays for Marked BVDV Vaccines

Cattle of various ages may be vaccinated with either a live-attenuated or inactivated NS3-mutated (marked) BVDV vaccine according to instructions provided. Serum samples can be collected 2-3 weeks or later following vaccination. To differentiate between cattle, which received the marked BVDV vaccine versus those infected by a field (wild type) strain of BVDV, serum samples may be tested via a differential diagnostic assay. The NS3 protein with epitope-specific amino acid mutations can, when presented to cattle in the context of a marked vaccine, elicit the production of specific antibodies which will bind to the mutated epitopes of NS3 protein, but not to the non-mutated epitopes present on wild type virus. In the context of wild-type virus, the opposite is true—that specific antibodies may recognize the wild-type epitopes on the NS3 protein, but not the mutated form. Methods of assaying for antibody binding specificity and affinity are well known in the art, and include but are not limited to immunoassay formats such as ELISA, competitive immunoassays, radioimmunoassays, Western blots, indirect immunofluorescent assays, and the like.

A competitive ELISA may be an indirect or a direct assay. One example of a direct competitive assay is described herein. Whole or partial wild type viral antigens, including the NS3 protein (naturally or synthetically derived), may be used as an antigen source. Following coating of the ELISA plate with antigen under alkaline conditions, cattle serum samples and dilutions are added together with an optimized dilution of the epitope-specific mAb, and incubated for 30-90 min. Either horseradish peroxidase or alkaline phosphatase has been conjugated to the mAb to allow for colorimetric detection of binding. Following washing of the plates, an enzyme-specific chromogenic substrate is added, and after a final incubation step, the optical density of each well is measured at a wavelength appropriate for the substrate used. Depending on the level of reactivity of the cattle serum with the NS3 protein coating the plate, binding of the labeled mAb could be inhibited. A lack of binding by the mAb indicates the presence of antibodies in the cattle serum that recognize the wild type-specific epitope, indicative of a natural (wild-type) infection. In contrast, serum from cattle immunized with the marked vaccine possessing an epitope specific mutation(s) will not contain antibodies which will bind to the NS3 protein coating the plate. Therefore, the mAb will bind to the NS3 protein, and result in subsequent color development.

Numerous variations will occur to those skilled in the art in light of the foregoing disclosure. For example, other cytopathic strains of BVDV may be mutated in the helicase domain of NS3 in a manner analogous to that exemplified herein by the NADL strain. While the exemplary mutations herein use alanine, other non-conservative amino acid replacements, or other mutations resulting in the retention of replication but the loss of recognition by antibodies raised to wild-type NS3 are within the purview of the invention. These are merely exemplary.

1. A bovine viral diarrhea virus comprising at least one helicase domain amino acid mutation wherein at least one mutation is within the SES loop of the helicase domain of NS3 and results in reduced binding by a monoclonal antibody raised against NS3 from wild-type bovine viral diarrhea virus but wherein viral RNA replication and the generation of infectious virus is retained.

2. The bovine viral diarrhea virus of claim 1, wherein the monoclonal antibody is selected from the group consisting of 20.10.6; 1.11.3; 21.5.8; and 24.8.

3. The bovine viral diarrhea virus of claim 1 wherein the virus comprises a single helicase domain amino acid mutation.

4. The bovine viral diarrhea virus of claim 1 comprising a helicase domain mutation within the IGR loop.

5. The bovine viral diarrhea virus of claim 4 comprising a helicase domain mutation within the IGR loop at amino acid residue 1841.

6. The bovine viral diarrhea virus of claim 4 comprising a helicase domain mutation within the IGR loop at amino acid residue 1843.

7. The bovine viral diarrhea virus of claim 4 comprising a helicase domain mutation within the IGR loop at amino acid residue 1845.

8. The bovine viral diarrhea virus of claim 1 comprising a helicase domain mutation within the KHP loop.

9. The bovine viral diarrhea virus of claim 8 comprising a helicase domain mutation within the KHP loop at amino acid residue 1867.

10. The bovine viral diarrhea virus of claim 8 comprising a helicase domain mutation within the KHP loop at amino acid residue 1868.

11. The bovine viral diarrhea virus of claim 8 comprising a helicase domain mutation within the KHP loop at amino acid residue 1869.

12. The bovine viral diarrhea virus of claim 1 comprising a helicase domain mutation within the SES loop.

13. The bovine viral diarrhea virus of claim 3, wherein the helicase domain mutation corresponds to at amino acid residue 1939 of SEQ ID NO: 1.

14. The bovine viral diarrhea virus of claim 3, wherein the helicase domain mutation corresponds to amino acid residue 1942 of SEQ ID NO: 1.

15. The bovine viral diarrhea virus of claim 1 wherein the virus comprises two, three, or four helicase domain amino acid mutations.

16. The bovine viral diarrhea virus of claim 15 comprising two helicase domain mutations.

17. The bovine viral diarrhea virus of claim 16 wherein the two helicase domain mutations are within the IGR loop.

18. The bovine viral diarrhea virus of claim 17 wherein the two helicase domain mutations within the IGR loop are at amino acid residues 1843 and 1845.

19. The bovine viral diarrhea virus of claim 16 wherein the two helicase domain mutations are within the SES loop.

20. The bovine viral diarrhea virus of claim 19 wherein the two helicase domain mutations within the SES loop correspond to amino acid residues 1939 and 1942 of SEQ ID NO: 1.

21. The bovine viral diarrhea virus of claim 15 comprising three helicase domain mutations.

22. The bovine viral diarrhea virus of claim 21 wherein the three helicase domain mutations are within the KHP loop.

23. The bovine viral diarrhea virus of claim 22 wherein the three helicase domain mutations within the KHP loop correspond to amino acid residues 1867, 1868, and 1869.

24. The bovine viral diarrhea virus of claim 21, wherein the three helicase domain mutations are within the IGR loop, the KHP loop, and the SES loop correspond to amino acid residues 1845, 1868, and 1939 of SEQ ID NO: 1.

25. A marked bovine viral diarrhea virus vaccine comprising the bovine viral diarrhea virus of claim 1.

26. A method of differentiating an animal infected with bovine diarrhea virus from an animal vaccinated with a bovine diarrhea virus vaccine wherein said bovine diarrhea virus vaccine is a marked vaccine comprising at least one helicase domain amino acid mutation, said method comprising;
(a) obtaining a test sample from a test animal;
(b) detecting bovine diarrhea virus in said test sample; and
(c) determining whether the bovine diarrhea virus contains the mutation.

27. The method of claim 26 wherein said method of detecting bovine diarrhea virus employs the use of at least one monoclonal antibody.

28. The method of claim 26 wherein the marked vaccine helicase domain amino acid mutation is in the helicase domain of NS3.

29. The method of claim 27 comprising the steps of:
(a) adding labeled antibody capable of detecting wild type bovine diarrhea virus or capable of detecting mutated bovine diarrhea virus to a test sample, wherein the test sample contains body fluid from test animal and;
(b) measuring the binding affinity of said labeled antibody to said wild type bovine diarrhea virus or to said mutated bovine diarrhea virus by contacting at least one monoclonal antibody to said wild type bovine diarrhea virus or to said mutated bovine diarrhea virus; and
(c) determining the vaccination status of test animal by comparing results of binding affinity using a monoclonal antibody directed to wild type BVDV versus BVDV with mutated NS3.

30. The method of claim 27 comprising the steps of:
(a) adding a labeled first antibody directed to a domain other than mutated NS3; and
(b) adding a labeled second antibody directed to a mutated portion of NS3.

31. The method of claim 30 wherein the first antibody is directed to a wild type virus.

32. The method of claim 30 wherein the second antibody is directed to the mutated portion of NS3.

33. The method of claim 32 wherein the second antibody is directed against NS3 and is selected from the group consisting of 20.10.6; 1.11.3; 21.5.8; and 24.8.

34. The method of claim 32 wherein the second antibody is directed to at least one mutated portion of the NS3 selected from the group consisting of the IGR loop, the KHP loop, and the SES loop.

35. The method of claim 34 wherein the bovine viral diarrhea virus comprises at least one helicase domain amino acid mutation within the IGR loop at an amino acid residue selected from the group consisting of 1841, 1843, and 1845.

36. The method of claim 34 wherein the bovine viral diarrhea virus comprises at least one helicase domain amino acid mutation within the KHP loop at an amino acid residue selected from the group consisting of 1867, 1868, and 1869.

37. The method of claim 34 wherein the bovine viral diarrhea virus comprises of at least one helicase domain amino acid mutation within the SES loop at an amino acid residue selected from the group consisting of 1939, and 1942.

38. The method of claim 34 wherein the bovine viral diarrhea virus comprises at least one helicase domain amino acid mutation within the IGR loop, the KHP loop, and the SES loop at amino acid residues 1845, 1868, and 1939.


SEQUENCE LISTING (21 sequences)

SEQ ID NO: 1 - protein, 3906 aa, Artificial
  Artificial bovine diarrhea virus polyprotein. Unprocessed polypeptide from RNA template for BVD virus, NADL isolate
LKPGPLFYQDYKGPVYHRAPLELFEEGSMCETTKRIGRVTGSDGKLYHIYVCIDGCIIIKSATRSYQRVFRWVHNRLDCPLWVTTCSDTKEEGATKKKTQKPDRLERGKMKIVPKESEKDSKTKPPDATIVVEGVKYQVRKKGKTKSKNTQDGLYHNKNKPQESRKKLEKALLAWAIIAIVLFQVTMGENITQWNLQDNGTEGIQRAMFQRGVNRSLHGIWPEKICTGVPSHLATDIELKTIHGMMDASEKTNYTCCRLQRHEWNKHGWCNWYNIEPWILVMNRTQANLTEGQPPRECAVTCRYDRASDLNVVTQARDSPTPLTGCKKGKNFSFAGILMRGPCNFEIAASDVLFKEHERISMFQDTTLYLVDGLTNSLEGARQGTAKLTTWLGKQLGILGKKLENKSKTWFGAYAASPYCDVDRKIGYIWYTKNCTPACLPKNTKIVGPGKFGTNAEDGKILHEMGGHLSEVLLLSLVVLSDFAPETASVMYLILHFSIPQSHVDVMDCDKTQLNLTVELTTAEVIPGSVWNLGKYVCIRPNWWPYETTVVLAFEEVSQVVKLVLRALRDLTRIWNAATTTAFLVCLVKIVRGQMVQGILWLLLITGVQGHLDCKPEFSYAIAKDERIGQLGAEGLTTTWKEYSPGMKLEDTMVIAWCEDGKLMYLQRCTRETRYLAILHTRALPTSVVFKKLFDGRKQEDVVEMNDNFEFGLCPCDAKPIVRGKFNTTLLNGPAFQMVCPIGWTGTVSCTSFNMDTLATTVVRTYRRSKPFPHRQGCITQKNLGEDLHNCILGGNWTCVPGDQLLYKGGSIESCKWCGYQFKESEGLPHYPIGKCKLENETGYRLVDSTSCNREGVAIVPQGTLKCKIGKTTVQVIAMDTKLGPMPCRPYEIISSEGPVEKTACTFNYTKTLKNKYFEPRDSYFQQYMLKGEYQYWFDLEVTDHHRDYFAESILVVVVALLGGRYVLWLLVTYMVLSEQKALGIQYGSGEVVMMGNLLTHNNIEVVTYFLLLYLLLREESVKKWVLLLYHILVVHPIKSVIVILLMIGDVVKADSGGQEYLGKIDLCFTTVVLIVIGLIIARRDPTIVPLVTIMAALRVTELTHQPGVDIAVAVMTITLLMVSYVTDYFRYKKWLQCILSLVSAVFLIRSLIYLGRIEMPEVTIPNWRPLTLILLYLISTTIVTRWKVDVAGLLLQCVPILLLVTTLWADFLTLILILPTYELVKLYYLKTVRTDTERSWLGGIDYTRVDSIYDVDESGEGVYLFPSRQKAQGNFSILLPLIKATLISCVSSKWQLIYMSYLTLDFMYYMHRKVIEEISGGTNIISRLVAALIELNWSMEEEESKGLKKFYLLSGRLRNLIIKHKVRNETVASWYGEEEVYGMPKIMTIIKASTLSKSRHCIICTVCEGREWKGGTCPKCGRHGKPITCGMSLADFEERHYKRIFIREGNFEGMCSRCQGKHRRFEMDREPKSARYCAECNRLHPAEEGDFWAESSMLGLKITYFALMDGKVYDITEWAGCQRVGISPDTHRVPCHISFGSRMPFRQEYNGFVQYTARGQLFLRNLPVLATKVKMLMVGNLGEEIGNLEHLGWILRGPAVCKKITEHEKCHINILDKLTAFFGIMPRGTTPRAPVRFPTSLLKVRRGLETAWAYTHQGGISSVDHVTAGKDLLVCDSMGRTRVVCQSNNRLTDETEYGVKTDSGCPDGARCYVLNPEAVNISGSKGAVVHLQKTGGEFTCVTASGTPAFFDLKNLKGWSGLPIFEASSGRVVGRVKVGKNEESKPTKIMSGIQTVSKNRADLTEMVKKITSMNRGDFKQITLATGAGKTTELPKAVIEEIGRHKRVLVLIPLRAAAESVYQYMRLKHPSISFNLRIGDMKEGDMATGITYASYGYFCQMPQPKLRAAMVEYSYIFLDEYHCATPEQLAIIGKIHRFSESIRVVAMTATPAGSVTTTGQKHPIEEFIAPEVMKGEDLGSQFLDIAGLKIPVDEMKGNMLVFVPTRNMAVEVAKKLKAKGYNSGYYYSGEDPANLRVVTSQSPYVIVATNAIESGVTLPDLDTVIDTGLKCEKRVRVSSKIPFIVTGLKRMAVTVGEQAQRRGRVGRVKPGRYYRSQETATGSKDYHYDLLQAQRYGIEDGINVTKSFREMNYDWSLYEEDSLLITQLEILNNLLISEDLPAAVKNIMARTDHPEPIQLAYNSYEVQVPVLFPKIRNGEVTDTYENYSFLNARKLGEDVPVYIYATEDEDLAVDLLGLDWPDPGNQQVVETGKALKQVTGLSSAENALLVALFGYVGYQALSKRHVPMITDIYTIEDQRLEDTTHLQYAPNAIKTDGTETELKELASGDVEKIMGAISDYAAGGLEFVKSQAEKIKTAPLFKENAEAAKGYVQKFIDSLIENKEEIIRYGLWGTHTALYKSIAARLGHETAFATLVLKWLAFGGESVSDHVKQAAVDLVVYYVMNKPSFPGDSETQQEGRRFVASLFISALATYTYKTWNYHNLSKVVEPALAYLPYATSALKMFTPTRLESVVILSTTIYKTYLSIRKGKSDGLLGTGISAAMEILSQNPVSVGISVMLGVGAIAAHNAIESSEQKRTLLMKVFVKNFLDQAATDELVKENPEKIIMALFEAVQTIGNPLRLIYHLYGVYYKGWEAKELSERTAGRNLFTLIMFEAFELLGMDSQGKIRNLSGNYILDLIYGLHKQINRGLKKMVLGWAPAPFSCDWTPSDERIRLPTDNYLRVETRCPCGYEMKAFKNVGGKLTKVEESGPFLCRNRPGRGPVNYRVTKYYDDNLREIKPVAKLEGQVEHYYKGVTAKIDYSKGKMLLATDKWEVEHGVITRLAKRYTGVGFNGAYLGDEPNHRALVERDCATITKNTVQFLKMKKGCAFTYDLTISNLTRLIELVHRNNLEEKEIPTATVTTWLAYTFVNEDVGTIKPVLGERVIPDPVVDINLQPEVQVDTSEVGITIIGRETLMTTGVTPVLEKVEPDASDNQNSVKIGLDEGNYPGPGIQTHTLTEEIHNRDARPFIMILGSRNSISNRAKTARNINLYTGNDPREIRDLMAAGRMLVVALRDVDPELSEMVDFKGTFLDREALEALSLGQPKPKQVTKEAVRNLIEQKKDVEIPNWFASDDPVFLEVALKNDKYYLVGDVGELKDQAKALGATDQTRIIKEVGSRTYAMKLSSWFLKASNKQMSLTPLFEELLLRCPPATKSNKGHMASAYQLAQGNWEPLGCGVHLGTIPARRVKIHPYEAYLKLKDFIEEEEKKPRVKDTVIREHNKWILKKIRFQGNLNTKKMLNPGKLSEQLDREGRKRNIYNHQIGTIMSSAGIRLEKLPIVRAQTDTKTFHEAIRDKIDKSENRQNPELHNKLLEIFHTIAQPTLKHTYGEVTWEQLEAGVNRKGAAGFLEKKNIGEVLDSEKHLVEQLVRDLKAGRKIKYYETAIPKNEKRDVSDDWQAGDLVVEKRPRVIQYPEAKTRLAITKVMYNWVKQQPVVIPGYEGKTPLFNIFDKVRKEWDSFNEPVAVSFDTKAWDTQVTSKDLQLIGEIQKYYYKKEWHKFIDTITDHMTEVPVITADGEVYIRNGQRGSGQPDTSAGNSMLNVLTMMYGFCESTGVPYKSFNRVARIHVCGDDGFLITEKGLGLKFANKGMQILHEAGKPQKITEGEKMKVAYRFEDIEFCSHTPVPVRWSDNTSSHMAGRDTAVILSKMATRLDSSGERGTTAYEKAVAFSFLLMYSWNPLVRRICLLVLSQQPETDPSKHATYYYKGDPIGAYKDVIGRNLSELKRTGFEKLANLNLSLSTLGVWTKHTSKRIIQDCVAIGKEEGNWLVKPDRLISSKTGHLYIPDKGFTLQGKHYEQLQLRTETNPVMGVGTERYKLGPIVNLLLRRLKILLMTAVGVSS

SEQ ID NO: 2 - DNA, 18 bp, Artificial
  Primer - Flanks 5′ end of p15aDI cloning site for mutant fragments
gaggccgttaacatatca

SEQ ID NO: 3 - DNA, 27 bp, Artificial
  Primer - Flanks 3′ end of p15aDI cloning site for mutant fragments
cctaaatcactttgaccctgttgctgt

SEQ ID NO: 4 - DNA, 30 bp, Artificial
  Primer - 5′ primer for introducing I1841A mutation
gaggcagggcgccacaagagagtattagtt

SEQ ID NO: 5 - DNA, 32 bp, Artificial
  3′ primer for introducing I1841A mutation
cttgtggcgccctgcctcctctataactgctt

SEQ ID NO: 6 - DNA, 30 bp, Artificial
  5′ primer for introducing R1843A mutation
gagataggcgcccacaagagagtattagtt

SEQ ID NO: 7 - DNA, 27 bp, Artificial
  3′ primer for introducing R1843A mutation
cttgtgggcgcctatctcctctataac

SEQ ID NO: 8 - DNA, 32 bp, Artificial
  5′ primer for introducing K1845A mutation
atagggcgccacgcgagagtattagttcttat

SEQ ID NO: 9 - DNA, 30 bp, Artificial
  3′ primer for introducing K1845A mutation
tctcgcgtggcgccctatctcctctataac

SEQ ID NO: 10 - DNA, 34 bp, Artificial
  5′ primer for introducing K1867A mutation
ttggctcacccatcgatctcttttaacctaagga

SEQ ID NO: 11 - DNA, 37 bp, Artificial
  3′ primer for introducing K1867A mutation
agagatcgatgggtgagccaatctcatatactggtag

SEQ ID NO: 12 - DNA, 31 bp, Artificial
  5′ primer for introducing H1868A mutation
aaagctccatcgatctcttttaacctaagga

SEQ ID NO: 13 - DNA, 34 bp, Artificial
  3′ primer for introducing H1868A mutation
agagatcgatggagctttcaatctcatatactgg

SEQ ID NO: 14 - DNA, 34 bp, Artificial
  5′ primer for introducing P1869A mutation
cacgcgagcataagctttaacctaaggatagggg

SEQ ID NO: 15 - DNA, 36 bp, Artificial
  3′ primer for introducing P1869A mutation
ttaaagcttatgctcgcgtgtttcaatctcatatac

SEQ ID NO: 16 - DNA, 32 bp, Artificial
  5′ primer for introducing E1939A mutation
ccatcgattttcagcgagtataagggttgtcg

SEQ ID NO: 17 - DNA, 32 bp, Artificial
  3′ primer for introducing E1939A mutation
ctcgctgaaaatcgatggatcttcccgataat

SEQ ID NO: 18 - DNA, 42 bp, Artificial
  5′ primer for introducing R1942A mutation
ccatcgattttcagagagtatagcggttgtcgccatgactgc

SEQ ID NO: 19 - DNA, 41 bp, Artificial
  3′ primer for introducing R1942A mutation
accgctatactctctgaaaatcgatggatcttcccgataat

SEQ ID NO: 20 - protein, 166 aa, Artificial
  Fragment of NS3 domain of BVDV
SKNRADLTEMVKKITSMNRGDFKQITLATGAGKTTELPKAVIEEIGRHKRVLVLIPLRAAAESVYQYMRLKHPSISFNLRIGDMKEGDMATGITYASYGYFCQMPQPKLRAAMVEYSYIFLDEYHCATPEQLAIIGKIHRFSESIRVVAMTATPAGSVTTTGQKHP

SEQ ID NO: 21 - protein, 145 aa, Artificial
  Fragment of NS3 domain of HCV
PPAVPQTFQVAHLHAPTGSGKSTKVPAAYAAQGYKVLVLNPSVAATLGFGVYMSKAHGIDPNIRTGVRAITTGGPITYSTYGKFLADGGCSGGAYDIIICDECHSTDSTSILGIGTVLDQAETAGARLVVLATATPPGSITVPHP